(19) United States
(12) Patent Application Publication
(10) Pub. No.: US 2005/0165327 A1
(43) Pub. Date: Jul. 28, 2005

(54) Apparatus and method for detecting the severity of brain function impairment

(76) Inventors: Lawrence E. Thibault, Chadds Ford, PA (US); Kirk L. Thibault, Berwyn, PA (US); James L. Thibault, Philadelphia, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/766,458

(22) Filed: Jan. 28, 2004

(51) Int. Cl. A61B 13/00; A61B 5/00

(52) U.S. Cl. 600/558; 600/559

(57) ABSTRACT

A method for determining the severity of brain function impairment in a person due to an insult to the brain such as mild traumatic brain injury, aging or intoxication is disclosed including the steps of determining a processing time value of the person in accordance with an elapsed time between a stimulus applied to the person and a response to the stimulus provided by the person, wherein the processing time value is representative of the impaired brain function. Electrical signals are provided in accordance with the processing time value, whereby the electrical signals are representative of the impaired brain function. Mathematical operations are performed upon the electrical signals to provide a processing time index. The severity of brain function impairment is determined in accordance with the processing time index. The severity of a concussion or brain impairment can be determined quantitatively in accordance with the processing time index.

### BACKGROUND OF THE INVENTION

1. Field of Invention

This invention relates to a system and method for detecting impairment of brain function and, in particular, for detecting impairment of brain function due to mild traumatic brain injury, aging or intoxication.

2. Description of Related Art

Central nervous system injury remains among the top priorities of the Nation's health care research agenda. Minor head injury or mild traumatic brain injury (MTBI) accounts for the majority of the estimated 2,000,000 brain injuries that occur each year in the United States. This type of brain injury is at the low-end of the continuous spectrum of Diffuse Axonal Injury (DAI), and currently there is no quantitative measure of this form of brain injury with the sensitivity that is required for further study of injury mechanisms, diagnosis, functional impairment or recovery. While it is known in the art to make subjective judgements, for example on the speed of retinal responses to light applied to the eye of a subject, such inexact subjective judgements lack the value of a more quantitative measurement. Although such a quantitative measure of this form of brain injury is lacking, many other measurements of brain function are known in the art.

For example, U.S. Pat. No. 4,755,043 issued to Carter on Jul. 5, 1988, entitled “Portable Standing Digital Pupillometer and Method of Use Thereof,” discloses a portable hand-held dynamic automatic scanning, measuring and recording device capable of inspecting holes and measuring gaps in animate or inanimate objects up to 10 millimeters in size, such as the pupil of a vertebrate eye or a printed circuit board. The device included a lightweight viewing optics/image sensor and a microprocessor-controlled, automatic scanning and data/storage system with a digital readout of pupil diameter measurements.

U.S. Pat. No. 4,850,691 issued to Gardner, et al., on Jul. 25, 1988, entitled “Method and Apparatus for Determining Pupillary Response Parameters,” discloses a method and apparatus for determining pupillary response parameters, such as: latency, time of acceleration of constriction, and speed of constriction. In a form of the disclosed method, a signal was derived which was representative of measured pupil size as a function of time after a visible light stimulus. Pupillary response parameters could be obtained from the modified signal. The modification of the signal included fitting curves to different portions of the signal, correcting at least one of the fitted curves, and deriving the modified signal from the curves, as corrected.

U.S. Pat. No. 5,746,205 issued to Virsu, on May 5, 1998, entitled “Method and Apparatus for Measuring the Working Condition of the Brain with Periodic Stimuli,” discloses a system wherein the working quality of the human brain was measured by determining the highest frequency at which the subject can distinguish between the synchronous and asynchronous pairs of two simultaneously presented periodic non-verbal stimuli; the higher the frequency “synchronization threshold,” the faster the brain of the subject can process information using the sensory function tested.

U.S. Pat. No. 5,956,125 issued to Rosse on Sep. 21, 1999, entitled “System and Method for Screening for Dementia,” teaches a system and method for screening persons for dementia, particularly dementia of the Alzheimer's type. The system and method included means for radiating the eye of a person with light, preferably light solely within the infrared spectrum, and means for generating a signal responsive to the amount of light reflected from the eye over a period of time. The signal was taught as indicative of changes in the pupils size over time. The system and method also included a means for performing a Fourier transform upon the signal to provide a value indicative of the strength of this signal's frequency component.

U.S. Pat. No. 6,024,707 issued to Scinto, et al., on Feb. 15, 2000, et al, entitled “Non-Invasive Method for Diagnosing Alzheimer's Disease in a Patient,” discloses a non-invasive method for diagnosing Alzheimer's disease in a living human subject. The method included a non-invasive automated apparatus which could continuously monitor pupil diameter size over time, repetitively measure pupil diameter size over time for a pre-chosen duration ranging from about less than one second to about five minutes, and cumulatively record size information as it was obtained over time. Another method disclosed by Scinto, et al., employed an apparatus which repetitively measured pupil constriction velocity for a pre-chosen duration both before and after stimulation by visible light.

U.S. Pat. No. 6,097,295 issued to Griesinger, et al., on Aug. 1, 2000, entitled “Apparatus for Determining the Alertness of a Driver,” discloses an apparatus for determining the alertness of a person, especially a vehicle or machine operator. The apparatus taught by Griesinger included an image pick-up system for recording images in the area of at least one eye of the person and an image evaluation system that contained means for detecting the shutting of an eye. The image evaluating system had means for determining pupil size and an evaluation apparatus that determined the alertness according to the eye closing state information obtained by the closed-eye detection means. The determination was made according to the pupil size information determined by the pupil size determining means.

U.S. Pat. No. 6,199,985 issued to Anderson, on Mar. 3, 2001, entitled “Pupilometer Methods and Apparatus,” discloses an apparatus for detecting and measuring pupillary size and response to a light stimulus. The apparatus disclosed by Anderson included an infrared source and optical apparatus for directing an infrared beam toward an individual's pupil. The apparatus also included a measuring channel comprising optical and photo-optical detection apparatus for directing and receiving a first portion of the infrared illumination leaving the individual's pupil and for providing an electrical output signal indicating the illumination power detected. The calibration channel, including a second optical and photo-optical apparatus adapted to receive a second portion of the infrared illumination leaving the individual's eye, was also included for providing an electrical output signal indicative of certain individual-specific, pupillary, output parameters. The output signal from the measuring channel, providing information regarding relative pupillary size changes when the pupil was light-stimulated, was processed with the output signal lines from the calibration channel to provide actual or absolute pupillary size and response data.

U.S. Pat. No. 6,260,968 issued to Stark, et al., on Jul. 17, 2003, entitled “Pupilometer with Pupil Irregularity Detection Capability,” discloses the pupilometer having a pupil irregularity or non-uniformity detection capability. The pupilometer could include an imaging center for generating signals representative of the pupil of an eye, a data processor and a program executable by the data processor for enabling the data processor to thereby identify one or more regions of non-uniformity within the image of a perimeter of the pupil. The pupilometer included thresholding routines and could be coupled to a network containing a medical database and data processing hardware.

U.S. Pat. No. 6,385,486 issued to John, et al., on May 7, 2002, entitled “Brain Function Scan System,” discloses a portable elecrtroencephalograph instrument especially for use in emergencies and brain assessments in physician's offices. The device detected and amplified brain waves and converted them into digital data for analysis by comparison with data from normal groups. Electrodes in a headband were provided for broadcasting the data by radio or cellular phone to a local receiver for re-transmission or analysis.

U.S. Pat. No. 6,478,424 issued to Grinvald, et al., on Nov. 12, 2002, entitled “Non-Invasive Imaging of Retinal Function,” discloses a system for imaging reflectance changes and intrinsic fluorescence changes of a retina due to retinal function, including an imaging illuminator for illuminating the retina and a retina stimulating illuminator for inducing a functional response. The device also included an imaging apparatus for receiving light from the retina via retinal imaging objects, image acquisition means for digitizing and storing images from the imaging device, and a computer for controlling the operation of the system and for processing the stored images to reveal a differential functional signal corresponding to the retina's function.

U.S. Pat. No. 6,594,525 issued to Esteller, et al., on Jul. 15, 2003, entitled “Adaptive Method and Apparatus for Forecasting and Controlling Neurological Disturbances Under a Multi-Level Control,” discloses a method and apparatus for forecasting and controlling neurological abnormalities in humans such as seizures or other brain disturbances. The system was based on a multi-level control strategy. Using one or more types of physiological measurements as inputs such as, brain, electrical, chemical or magnetic activity, heart rate, pupil dilation, eye movement, temperature and chemical concentration of certain substances, a feature set was selected off-line from a pre-programmed feature library contained in a high level controller within a supervisory architecture. Supervisory control contained a knowledge base that was continuously updated with feed back information from an implantable device.

U.S. patent application Publication No. U.S. 200/0011250 A1, published by Stewart, et al., on Jan. 31, 2002, entitled “Procedure for Evaluated Vestibular Dysfunction,” teaches providing standardized guidelines for diagnosis and treatment of central nervous system abnormalities such as concussions, head trauma, memory loss, confusion or other cognitive dysfunction. An initial assessment was made of a person for loss of consciousness, spinal cord injury, or a secondary injury. The person was asked to perform a plurality of mental and physical exercises. A dynamic visual acuity test was also taken. The test could be performed on the sideline of an athletic playing field to determine whether it was advisable for an athlete to return to play. Additional tests, such as machine-based hearing, dynamic-vision and balance tests were administered to the individual. The results of the machine-based tests were transmitted electronically to an online data base and were evaluated.

European Patent Application EP 1122679 A2 published on Aug. 8, 2001, and having application number 01300830.5, filed on Jan. 31, 2001, is entitled “Neurobiological Pathology Diagnostic Apparatus and Methods”. The apparatus and method disclosed were for diagnosing the presence or absence of the symptoms of neurological pathology caused by a physical head trauma (such as occurs in contact sports or automobile collisions), disease (such as occurs in Alzheimer's disease), toxins (substance abuse or environmental toxins) or infection (such as occurs as a side effect of later stage AIDS infection). The apparatus and method were used for diagnosing neurological pathology, as well as for monitoring recovery from or maintenance of or progression of neurological pathology.

International Publication No. WO 01/54559 A2, published on Aug. 2, 2001 for International Application Number PCT/US01/02188 by Comrie, et al., entitled “Neurological Pathology Diagnostic Apparatus and Method,” discloses an apparatus and method for diagnosing the presence or absence of the symptoms of neurologic pathology. Memory was provided on a computer readable tangible media for storing neurological pathology testing protocols. Memory was also provided for storing responses to neurological pathology testing protocols and for storing user response analysis software. The response analysis software compared the neurologic pathology testing protocol results for the user with a baseline.

“Qualitative Pupillometry, A New Technology: Normative Data and Preliminary Observations in Patients with Acute Head Injury” by Taylor, et al, J. Neurosurg. 98:205-213, 2003 discloses a hand-held point and shoot pupilometer to assess pupillary functions quantitatively. Repetitive measurements were initially made in healthy volunteers providing a plurality of paired “alternative right eye, left eye” measurements under varying light conditions. Patients undergoing a variety of non-intracranial nonophthalmological endoscopic or surgical procedures such as patients in a cardiology clinic with acute head injury in whom intracranial pressure was continuously monitored were assessed. Additionally, patients suffering from subarachnoid hemorrhage were also studied. In certain patients, a reduction in constriction velocity was observed when either oral or intravenous narcotic agents and diazepam analogs were administered.

Other references illustrating the background of the present invention include: Denny-Brown D., Russell, W. R., Experimental Cerebral Concussion, Brain, 64:93-164, 1941; Holburn, A H S, Mechanics of brain injuries, Brit. Med. Bul. 3, pp. 147-9, 1445; Ommaya A. K. Hirsch A. E., Flamm E. S. and Mahone R. H., Cerebral concussion in the monkey: an experimental model, Science, 153:211-212, 1966; Ommaya, A., Gennarelli, T A, Cerebral concussion and traumatic unconsciousness., Brain 97, pp. 633-654, 1974; Gennarelli, T A Thibault, L E, Pathophysiologic responses of relational and translational accelerations of the head, Proceedings of the 16^{th} Stapp Car Crash Conference, SAE, pp. 296-308, 1972; Ommaya A. K., Hirsch A. E., and Martinez J. L., The role of whiplash in cerebral concussion, Proceedings of the 10^{th} Stapp Car Crash Conference, Nov., 1966; Gennarelli, T. A., Thibault, L. E., and Adams H., Diffuse Axonal Injury and Traumatic Coma in the Primate, Ann. Neurol., Vol. 12, pp564-574, 1982; Margulies, S. S. Biomechanics of Traumatic Coma in the Primate, Ph.D. Dissertation, University of Pennsylvania, 1987

All references cited herein are incorporated herein by reference in their entireties.

### BRIEF SUMMARY OF THE INVENTION

Central nervous system injury remains among the top priorities of the Nation's health care research agenda. Minor head injury or mild traumatic brain injury (MTBI) accounts for the majority of the estimated 2,000,000 brain injuries that occur each year in the United States. This type of brain injury is at the low-end of the continuous spectrum of Diffuse Axonal Injury (DAI), and currently there is no quantitative measure of this form of brain injury with the sensitivity that is required for further study of injury mechanisms, diagnosis, functional impairment or recovery. We believe that processing time, related to the anatomy affected by DAI, is an exquisitely sensitive measure of impairment in the injured patient. To this end, we are applying for a patent related to the design, development and testing of an apparatus-based system to measure the severity of a cerebral concussion and to track the recovery process. The apparatus-based system is designed to quantify the functional response of the central white matter in the brain by targeting and quantifying processing time through both active and passive apparatus-based tests. Through these tests, processing time measurements may be analyzed to calculate a novel, quantitative measure of impairment, the Processing Time Index (PTI). The PTI is a numerical representation of the level of functional impairment for an unhealthy individual compared to the healthy general population.

Experimental research has demonstrated that DAI results from trauma, in the form of dynamic stretch, to the axons of the deep central white matter within the brain. These axons respond to stretch by depolarizing concomitant to elevated intracellular calcium. Increasing levels of stretch result in graded forms of DAI from mild concussion to the persisting coma. The proposed system measures the reduction in responsiveness of the fiber tracts that are most affected by stretch and, by examining the response, automatically computes PTI. This system represents a new diagnostic tool for the neuropsychologist, emergency medical professional and other health care professionals who assess functional impairment and recovery associated with brain injury. This system is not simply a computerized version of existing neuropsychological testing, it is a set of apparatus delivering a novel set of tests and computing a numerical assessment of functional impairment in the form of the PTI.

The apparatus-based system is comprised of two distinct components:
1) Active Component: an interactive laptop computer-based system to administer a multi-modality test battery that measures a patient's processing time and computes the PTI. This system is considered “active” because it requires a conscious patient to interact with the system;
2) Passive Component: a hand-held, portable apparatus, henceforth referred to as the “Ocular Assessment Tool”, that the medical professional can employ to perform an automated pupillary reflex exam on a patient, quantify the results, and compute the PTI for the patient being examined. This system is considered “passive” because it does not require the patient to be conscious to administer the test.

The proposed computer test battery (active) can be administered in approximately ten minutes; the ocular assessment test (passive) can be administered in seconds, even to an unconscious patient. The ultimate applications of this compact, easy to use system, either as individual components or as a combined complementary system, include professional, collegiate, secondary school and “pee-wee” athletic and recreational environments, accidents on our highways, in industrial workplaces, in the military environment and in our homes. There are needs for such apparatuses in the arenas of emergency medicine, field medicine and the military. We also believe that the system may be useful in detecting impairment in certain non-trauma populations, namely the elderly and intoxicated, where impairment results not from injury but from other contributing factors that mimic the impairment in processing time seen in concussion (e.g., applications such as elderly driver's license exam, field sobriety tests, etc.).

In accordance with the present invention, it is believed that processing time, related to the anatomy affected by certain types of brain injury as well as non-traumatic mechanisms of impairment (e.g., toxins, disease), is a sensitive measure of neurological impairment in the unhealthy patient. Thus, the invention includes apparatus-based systems to measure the severity of functional neurological impairment and to track the recovery process of the patient. The apparatus-based systems of the invention are designed to quantify the functional response of the central white matter tracts in the brain by targeting and quantifying processing time through both active and passive apparatus-based tests. Through these tests, processing time measurements are analyzed in order to calculate quantitative measure of impairment, the PTI. The PTI can thus be used as a numerical representation of the level of functional impairment for an unhealthy individual compared to the healthy general population.

The systems of the invention measure the reduction in responsiveness of the fiber tracts of the axons that are most affected by stretch and, by examining the response, automatically compute PTI. The systems of the invention thus represent a diagnostic tool for the neuropsychologist, emergency medical professional and any other health care professionals who assess functional impairment and recovery associated with brain injury or other brain function impairment. In addition to providing a computerized version of appropriate existing neuropsychological testing, the systems of the present invention provide a novel set of apparatuses delivering a novel set of tests and computing a novel numerical assessment of functional impairment in the form of the PTI. The present systems are useful for determining the severity of impairment from non-traumatic factors related to, for example, aging and intoxication, as well as head trauma.

To determine PTI, two apparatus-based components, a laptop computer system and a handheld ocular assessment apparatus are proposed. The computer system is an “active” system with which the patient interacts. It is intended to record patient history and deliver a battery of automated multi-modality tests to measure processing time. The total test battery can be administered in approximately ten minutes. The ocular assessment is a “passive” system used by the medical professional to assess a patient's processing time via pupillary reflex examination. Administration of the ocular assessment examination is comparable to a traditional pupillary reflex exam; the test can be administered in seconds and the patient need not be conscious for the exam. Each test, either the active computer-based system or the passive ocular assessment, will result in the determination of a Processing Time Index, a numerical score that quantifies the level of impairment of the patient relative to the appropriate population norm.

The multi-apparatus system can be used as separate components or in concert, resulting in tremendous flexibility of application, from fixed facilities in emergency rooms and athletic training rooms, to field use in athletics, the emergency rescue services and military. The applications for the system range from injury evaluation and determination of acute impairment to tracking change in status and impairment during recovery and rehabilitation. There also exists potential for evaluating processing time impairment outside of the brain injury arena (e.g., the elderly or field sobriety testing).

Thus, a method for determining the severity of brain function impairment in a person due to an insult to the brain such as mild traumatic brain injury, aging or intoxication is disclosed including the steps of determining a processing time value of the person in accordance with an elapsed time between a stimulus applied to the person and a response to the stimulus provided by the person, wherein the deviation of the processing time value from normal is representative of the impaired brain function. Electrical signals are provided in accordance with the processing time value, whereby changes in the electrical signals from normal are representative of the impaired brain function. Mathematical operations are performed upon the electrical signals to provide a PTI. The severity of brain function impairment is determined in accordance with the processing time index.

A visual stimulus can be applied to the person and the severity of the brain function impairment can be determined in accordance with the visual stimulus. An audio stimulus can be applied to the person and the severity of the brain function impairment can be determined in accordance with the audio stimulus. A somatosensory stimulus can be applied to the person and the brain function impairment can be determined in accordance with the somatosensory stimulus. The brain function impairment can also be determined in accordance with any combination of the foregoing stimuli. Additionally, the severity of the brain function impairment can be determined using pupillometry, including by use of a hand held pupillometer known as an Ocular Assessment Tool (OAT).

### BRIEF DESCRIPTION OF SEVERAL VIEWS OF THE DRAWINGS

The invention will be described in conjunction with the following drawings in which like reference numerals designate like elements and wherein:

FIG. 1 is a schematic representation of the cascade of axonal injury resulting in impairment in processing time.

FIG. 2 is a block diagram of the conceptual framework for the processing time index in accordance with the proposed brain function impairment detection system.

FIG. 3 is a block diagram of the relationship between the active components and the passive components in determining the processing time index in accordance with the brain function impairment detection system.

FIG. 4 is a schematic diagram of the components of the active computer based system for determining processing time impairment.

FIG. 5 is a flow diagram of the active computer based brain function impairment detection system of the present invention of FIG. 4.

FIG. 6 is a neuroanatomical representation of the orthogonally oriented axonal tracks of the brain.

FIG. 7 is a sample target tracking test for use in accordance with the brain function impairment detection system of FIG. 4.

FIG. 8 is a graphical representation of a response to the target tracking test of FIG. 7.

FIG. 9 is a bar graph representation of a comparison of results obtained using the brain function impairment detection system of FIG. 4.

FIGS. 10A, B are schematic representations of two alternate embodiments of the passive component of the brain function impairment detection system in FIG. 3.

FIG. 11 is a flow diagram of a passive brain function impairment detection system such as the system of the present invention of FIG. 10A, B.

FIG. 12 is a schematic representation of the determination of pupil diameter using region of interest thresholding methods for the passive brain function impairment detection system of FIG. 10A, B.

FIG. 13 is a graphical representation of a response of the pupil generated by the passive component of the brain function impairment detection system of FIG. 10A, B.

### DETAILED DESCRIPTION OF THE INVENTION

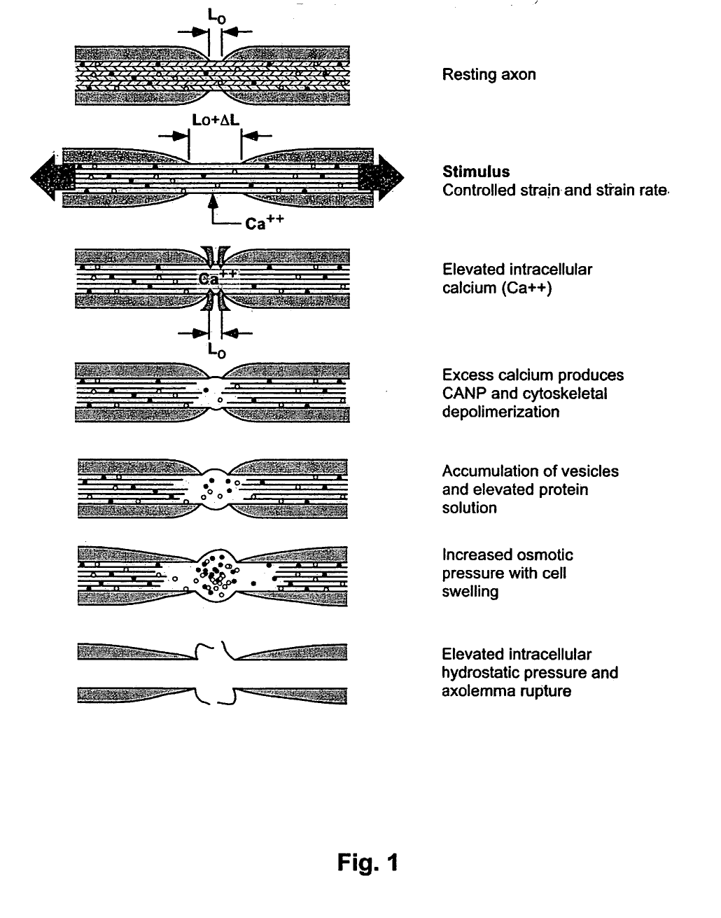

Referring now to FIG. 1, there is shown a plurality of views of an axon illustrating an injury cascade for understanding the differing responses to stimuli provided by healthy and injured axons. Study of this phenomenon has focused attention on Diffuse Axonal Injury (DAI) because epidemiological data indicated that these forms of injury are responsible for a large percentage of the mortality and morbidity associated with brain injury. By studying the mechanism of this type of brain injury with animal, physical and mathematical models, it is possible to estimate the magnitude and temporal nature of the deformations which are experienced by the various neural and neurovascular elements in association with these injuries. With this information, it is possible to investigate the biomechanics of injury at the isolated tissue and cellular levels in order to begin to simplify this complex analysis. Accordingly, this permits the designing of instrumentation that permits the study of isolated axons and cells in culture under conditions of controlled mechanical deformation. Utilizing these technologies, it has been determined that high strain rate of deformation of the axolemma leads to an elevated level of intracellular calcium.

Cell membrane ionic permeability is directly affected by high strain rate deformation that leads to an immediate elevation in cytosolic free calcium ion concentration. This traumatic rise in cytosotic free calcium in neurons has been implicated in cytoskeletal disruption, functional impairment, cell swelling, and ultimately cell death. Until the invention of the proposed system, there has been no direct, quantitative way to relate the magnitude of the strain at the level of the single cell to the complex portrait of brain injury and the various clinical manifestations; it is necessary to also know the anatomic distribution of the strain pattern in order to assess the global effects of injury to the whole brain. For example, the injuries, which are described as cerebral concussion, mild DAI, moderate DAI, and severe DAI with prolonged coma may represent the continuous spectrum of injury to the axons of the brain.

Processing time and impairment can be assessed quantitatively through the innovative approach of the proposed system. To this end, a time-based indicator of impairment is the most appropriate choice for expressing impairment. The determination of a PTI may be achieved through apparatuses that encompass a wide range of applications. The proposed system includes both an active apparatus (computer laptop based system) and a passive apparatus (hand held Ocular Assessment Tool for pupilometric measurements) that offer a wide range of flexibility and applicability across a myriad of potential patient and user environments.

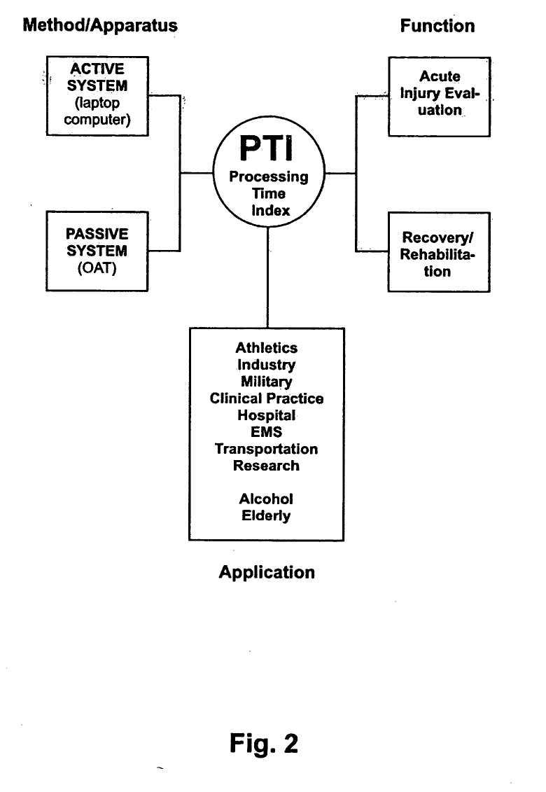

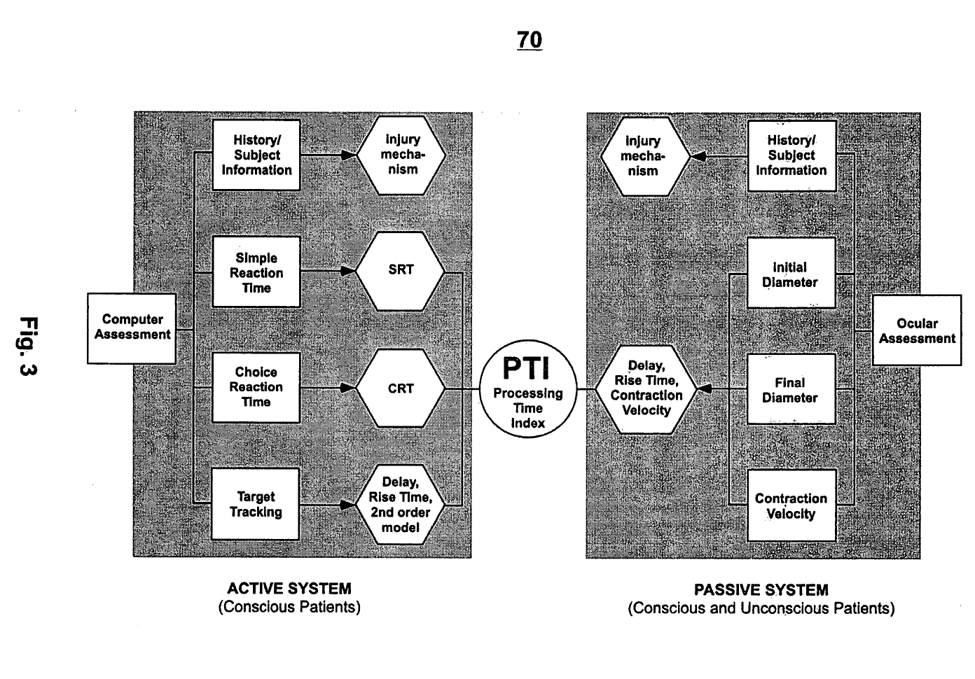

FIG. 2 shows an overall conceptual diagram of the relationship between the present system and its components and applications. FIG. 3 shows a schematic diagram representation 70 of the active components (e.g. the computer 16) of the brain function impairment detection system 10 for determining PTI as well as the passive components of a methodological and apparatus-based variation of the computer system 10, known as the Ocular Assessment Tool. Each component (the computer system 10 or the OAT) may be used separately or they may be used in concert, providing flexibility of application.

Active System (Computer-based Apparatus)

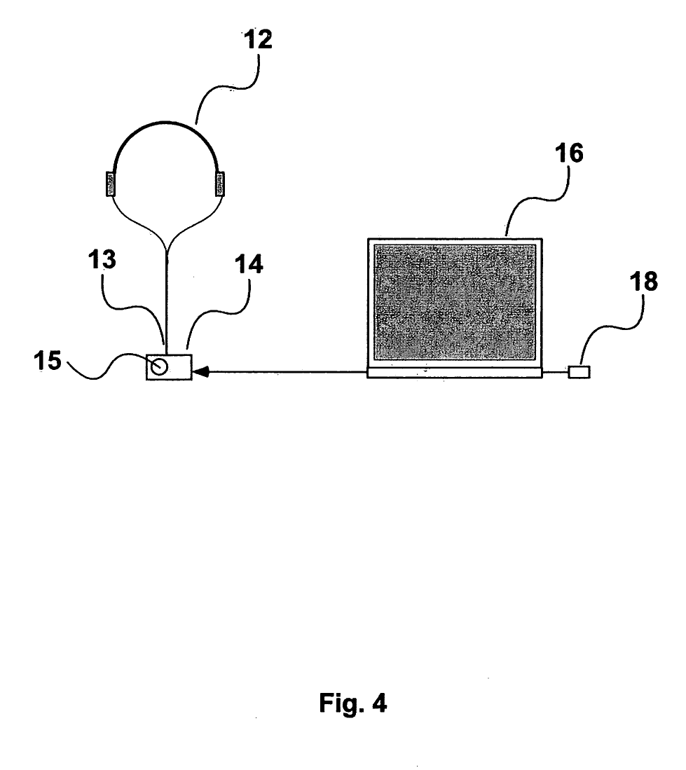

Referring now to FIG. 4, there is shown a schematic representation of the computer based brain function impairment detection system 10 of the present invention for determining the processing time index PTI to determine the severity of brain function impairment for cases of head trauma, aging and intoxication. The brain function impairment detection system 10 consists of a computer system 16 such as a laptop computer system and the custom hardware 14 and software. The computer system 16, which can be a laptop, PC or any other type of processor system, is a system with which the patient interacts. It requires the patient to be conscious and able to interact with the system, thus making it an “active” system. It is intended to record patient history and deliver a battery of automated tests measuring processing time of the patient. In a preferred embodiment of the invention, the total test battery of the brain function impairment detection system 10 can be administered in a relatively short period of time, for example, in ten to fifteen minutes or less. Delivery of the processing time test battery via the computer based system 10 results in a determination of the PTI, a numerical score that quantifies the level of impairment of the patient relative to a norm, such as an appropriate population norm.

The computer system 16 drives three types of stimuli for application to the patient: auditory, somatosensory (tactile) and visual. The visual stimuli can be delivered via a built-in display monitor of the computer system 16; auditory stimuli can be delivered via headphones 12 receiving an audio signal from a headphone jack 13 of the custom hardware 14. Somatosensory stimuli can be delivered by a touchpad 15 that can be essentially a small speaker coil or piezoelectric element that can be driven by the same audio signal as above; however, the audio signal vibrates the speaker coil or piezoelectric element, resulting in the patient's sensing of vibration on, for example, the patient's fingertip. In one embodiment of the invention the touchpad 15 can be disposed on the custom hardware 14 or finger clips (not shown) could be provided for tactile delivery. The custom hardware 14 thus can consist of a signal box and a touchpad 15 that accepts the audio signal from the computer 16 and routes the signal to the headphones 12 (for the auditory stimulus) or the touchpad 15 (for the vibrotactile stimulus).

The system 10 is intended to be compact and self-contained such that it presents tremendous flexibility of application, from fixed facilities in emergency rooms and athletic training rooms, to field use in the emergency rescue services and military. For example, a hand held embodiment of the system 10 is discussed below. The applications for the brain function impairment detection system 10 range from injury evaluation and determination of acute impairment, to tracking changes in status and impairment during recovery and rehabilitation, progressive aging, or regaining sobriety. Potential therefore also exists for evaluating processing time impairment outside of the brain injury arena (e.g., impairment in the elderly or intoxicated individuals in field sobriety testing).

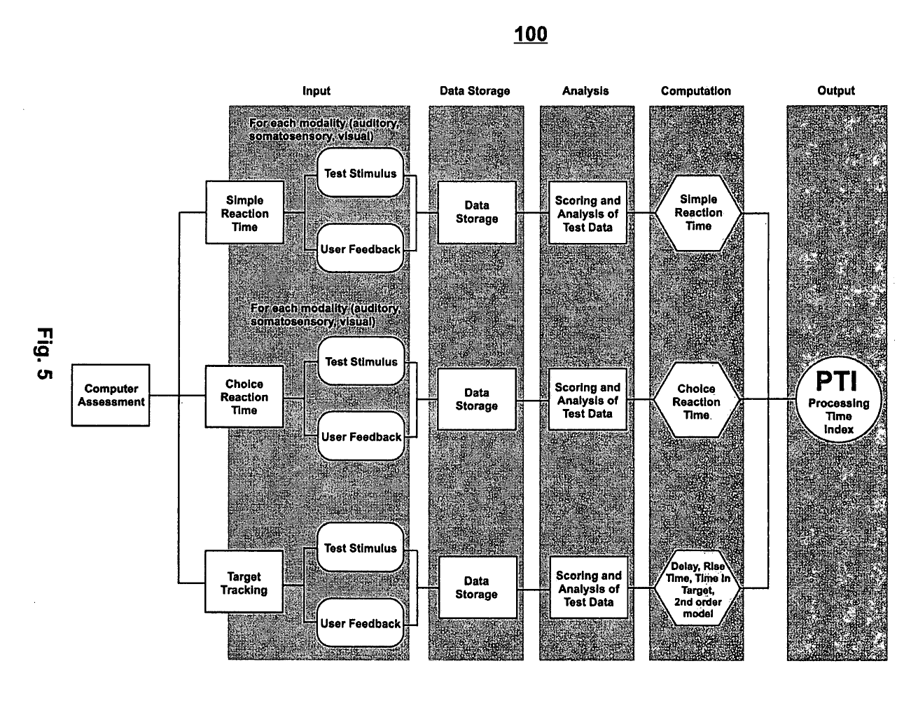

Referring now to FIG. 5, there is shown a block diagram representation 100 of an active computer-based method for delivering a test battery to a patient for determining PTI. In a preferred embodiment of the invention the test battery delivered according to the block diagram 100 can take approximately ten minutes as previously described. The method of the invention can extract the pertinent processing time data (such as simple reaction time, choice reaction time, delay, rise time and time in target) and automatically score an individual's PTI in accordance with the test battery of the block diagram representation 100.

It is believed that processing time as measured by the system 10 is the single most sensitive parameter that correlates strongly with concussion severity and impairment of brain function resulting therefrom or from other sources. Processing time and impairment are thus assessed quantitatively through the approach of the proposed brain injury detection system 10 using the conceptual framework 40 and the method set forth in the schematic diagram 70. To this end, a time-based indicator of impairment is the most appropriate choice for expressing impairment, namely the processing time index.

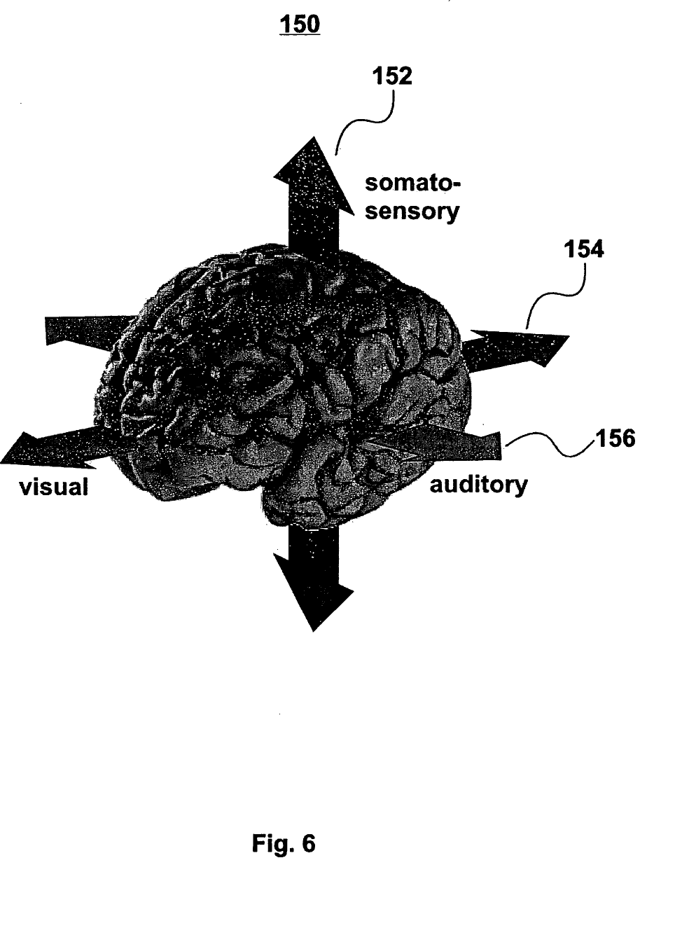

Referring now to FIG. 6, representation 150 illustrates that from a neuroanatomical standpoint the brain has three orthogonally oriented axonal tracts that may become impaired following an insult, such as a traumatic brain injury. The three orthogonal axes of the brain's major functional tracts are the auditory tract 156 (medial/lateral), the somatosensory tract 152 (superior/inferior) and the visual tract 154 (anterior/posterior). These three tracts correspond to the auditory, somatosensory and visual systems of the patient.

When the head experiences an impact or a purely inertial load the brain is free to move independently of the skull and to deform in proportion to the severity of the impact. The nerve fibers within the brain consist of bundles of axons, which experience stretch during this brain deformation. It has been demonstrated that the degree of stretch determines the changes seen in the electrophysiology of the axons. For small levels of stretch (less than approximately 5%) the membrane potential remains capable of propagating action potentials and the axons continue to function normally. At levels of stretch that are greater than approximately 5% the axon membranes have been shown to depolarize and have reduced capability to conduct until the membrane potential is restored. Calcium is at the heart of this problem, since it moves into the cytosol during the stretch through mechanically induced pore formation in the membrane. As the degree of stretch increases so does the level of calcium influx. This limits the ability to conduct information and the recovery process.

On the macroscopic level these processes are manifested as slowing in responses to stimuli (or an increase in processing time) or complete elimination of the nerve fiber response to external stimuli. Visual, auditory and somatosensory systems are incapable of processing information in the worst-case scenario. For lower levels of trauma or axonal strain, only a fraction of the conductors may be affected and therefore we see an increase in the processing time. In other words the impairment of the brain associated with concussion affects the areas of the major axonal tracts that transmit visual, auditory and somatosensory information.

To determine the severity of a cerebral concussion or other impairment, the compromises that have occurred as a result of the brain insult can be quantified by determining the PTI. This parameter will increase with increasing severity of impairment and can be followed through the recovery process.

The probing and measuring of the patient's temporal response to external stimuli is the concept behind the modern technique of multi-modality evoked potential (MMEP) testing. In conventional MMEP testing, each of the three tracts is stimulated individually and the electrical response to that stimulation is measured via surface electrodes attached to the patient's head. The actual response signal is typically small and embedded within electrical noise, requiring special cyclic stimulation/response measuring techniques and filtered averaging. In addition to several inconveniences to the patient, the MMEP testing process requires expensive, bulky dedicated hardware that is meant for fixed applications in a clinical setting. Nevertheless, the MMEP test can be a suitable test for probing the electrical activity of the brain to determine certain types of functional impairment.

However, the mobile, compact computer-based impairment detection system 10 of the invention can probe the same areas of the brain as the MMEP by introducing external stimuli specific to each anatomical region, and measuring the resulting processing time associated with the patient's response to the stimuli. The patient's response can be delivered directly to the impairment detection system 10 via a computer keyboard (not shown) coupled to the computer 16 and the mouse/stylus 18. The brain function impairment detection system 10 may be designed as a field tool with an Internet connection that permits remote access and collection of field data for examination by other medical professionals as well as researchers. This feature is easily implemented due to the base system being built on standard laptop computer technology and hardware.

The computer-based testing of the system 10 can be performed on the computer system 16 using algorithms and test methods developed within commercially available multi-media programming software (for example, Macromedia Director—Macromedia, Inc.) in a manner well understood by those skilled in the art. The test battery of system 10 can be presented in an intuitive, interactive way to provide user instructions in the user's choice of language (e.g., English or Spanish) text and speech. The software can allow complex multi-media content, thereby permitting the introduction of multi-lingual test instructions in textual, visual and verbal formats to make the test accessible to users with associated impairments or reading difficulties. Ultimately, remote collection and analysis of test data can require the end-user to have access to an Internet connection. Privacy and confidentiality of each test subject satisfies the new HIPAA requirements and is assured through the automatic generation of a unique identification number for each test subject. The identification number can be a combination of a machine ID, the test date and the sequential test subject number for the date. This identification method also permits retrospective identification of the test facility and date, should the need arise.

Because each test in the battery is focused on measuring a subject's processing time, time-based parameters can be automatically extracted from the test data and combined numerically to formulate a PTI within seconds of the test battery's completion. In one embodiment, five time-based measurements can constitute the input to the PTI: simple reaction time (SRT), choice reaction time (CRT), delay time, rise time, and time-in-target (from Target Tracking). The specific descriptions of the tests in the battery follow.

The simple and choice reaction time tests require the subject to respond to auditory, tactile or visual stimuli generated and applied to the subject by the computer 16. The response by the subject to the stimuli can be indicated by depressing a key, such as the space bar, on the keyboard of the computer 16 as quickly as possible. In the auditory simple reaction time test, the stimulus is a tone burst played through the headphones 12. The patient is instructed to react to the tone from headphones 12 as quickly as possible and to actuate an input device (e.g., keyboard, mouse button, touch screen) on the computer 16. In the somatosensory SRT, the tone burst signal is sent to a vibrotactile element (e.g., speaker coil/piezoelectric element 15) located on the custom hardware 14 or a separate embodiment (remote finger clip, not shown) upon which the patient's finger is resting. The patient is instructed to react when the vibrotactile stimulus is felt. In the visual SRT, the screen of the computer 16 can display an open, unfilled circle or other geometric shape. When the displayed unfilled circle changes to a filled circle (or changes from one color to another or changes to a predetermined size, or change any other such parameter as may be convenient) the subject responds. For SRT test modalities, the elapsed time between the stimulus and the response is recorded and stored automatically for further analysis.

In all SRT modalities, a practice test can be administered to familiarize the subject with the test process, followed by thirty trials of each modality recorded for analysis. The interval between each of the thirty trials can be randomly varied. Anticipating the stimulus and pressing the spacebar prematurely results in an error, the occurrence of which is recorded for further analysis. The test continues until all thirty trials have been administered and the data are stored on the computer 16 for analysis by the impairment detection system 10.

The choice reaction time (CRT) tests also require the subject to respond to auditory, tactile or visual stimuli generated by the computer; the response is achieved by depressing a key, such as the space bar, on the keyboard of the computer 16 as quickly as possible in response to the stimulus. In the auditory choice reaction time test, the stimulus is a tone burst played through the headphones 12. The patient is instructed to react to the tone as quickly as possible and press a key, such as the space bar, on the keyboard of the computer 16. Unlike the Simple Reaction Time test, the choice element can be to react only to the burst in a specified ear (perhaps the left ear only). In this case, if the tone burst plays in the right ear or both ears and a reaction is recorded, it is an error.

In the somatosensory CRT, the tone burst signal can be sent to a pair of vibrotactile elements (speaker coils or piezoelectric elements) on which the patient's fingers are resting, creating a buzzing vibration in one of a plurality of pads 15. The patient is instructed to react when they feel the vibration. As in the choice auditory test, the patient is instructed to react when the stimulus is in only one specific touch pad or other vibrotactile device 15 (perhaps the index finger pad 15 only). If the patient reacts incorrectly to the stimulus (in the other pad 15 or both pads 15), the response is recorded as an error for further analysis.

The visual CRT test is similar to the simple version, with the exception that a plurality, for example three, unfilled circles or other geometries are displayed, for example, side-by-side; the test subject is instructed to press a key, such as the space bar, on the keyboard of the computer 16 when the stimulus displayed results in only two of the three unfilled circles changing to filled circles. If the patient reacts to any other condition, an error is recorded.

In all CRT test modalities, a practice test is administered to familiarize the subject with the test process, followed by thirty trials of each modality recorded for analysis. The interval between each of the thirty trials is randomly varied. Anticipating the stimulus and pressing the spacebar prematurely results in an error, the occurrence of which is recorded for further analysis. The test continues until all thirty trials have been administered and the data are stored on the computer 16 for analysis.

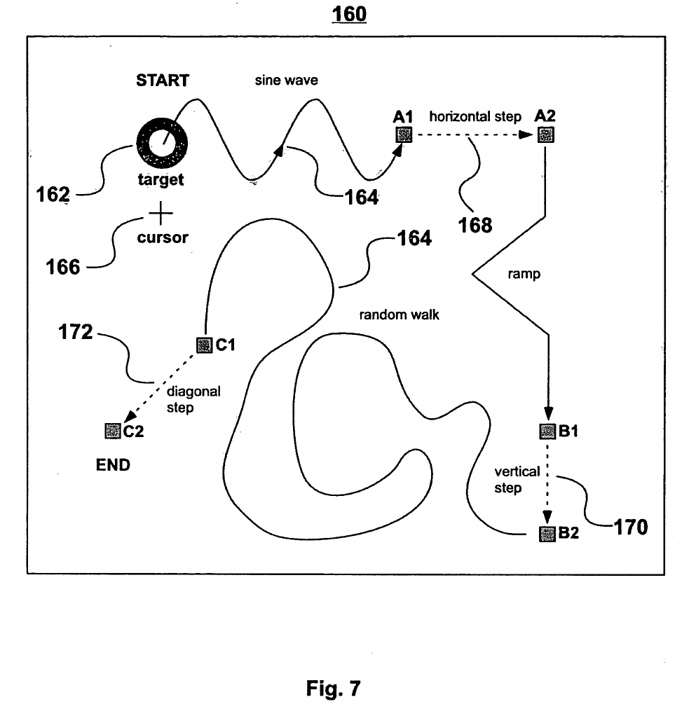

Referring now to FIG. 7, there is shown an example of a Target Tracking test 160 which can be used in cooperation with the method of the present intention. Target Tracking is the visual and somatosensory processing exercise that demands the test subject use the computer mouse 18 or a stylus or any other manual control device to control the motion and display of an icon such as a computer cursor 166 on the screen of the computer 16. The test consists of a target 162 on the display and the mouse 18 driven cross hairs of the cursor 166. The test subject is instructed to keep the cursor 166 inside the target 162 during the test by moving the cursor 166 in response to the movement of the target 162. The motion path 164 of the target 162 during the test is shown on the drawing for illustrative purposes only. The motion path 164 is not indicated on the computer 16 display viewed by the subject and is transparent to the subject throughout the entire test.

Initially, subjects are permitted practice to familiarize themselves with the cursor 166 movement as a function of their input via the mouse 18, stylus or touch-sensitive sceen. A brief practice session with the moving target 162 is administered to familiarize the subject with the goal of the test. The subject can initiate the actual test following the practice period by moving the cursor 166 into the stationary target 162. When this condition occurs, the target 162 begins its movement along the path 164 and the subject tracks the movement as closely as possible to completion of the test.

When the target 162 assumes motion that is prescribed by analytical or arbitrary mathematical expressions (forcing functions), the subject's response to the target 162 motion may be compared to the predicted solutions of those input forcing functions. The similarity between the subject's response and the response of a second order linear system makes this mathematical model a natural choice for fitting the subject's data. For example, previous experiments conducted on players in the National Football League have demonstrated a player's response to one type of target motion, prescribed as a step function, or an instantaneous change in location of the target 162, such as the step functions shown as steps 168, 170, 172 on target tracking test 160. The subject's response to tracking that motion is representative of a damped oscillatory response characteristic of a second order linear system. These changes in performance would be captured quantitatively in changes in the mathematical model of the player, represented by a second-order linear system fit. This mathematical model is an appropriate model that is easily solved, automated and fit.

In general, the differential form of the second order linear system can be expressed as follows:
\ddot{x}+A\dot{x}+Bx=F(t)
where the dot notation represents the n^{th} derivative of x with respect to time, and the forcing function F is also a function of time t. When this equation is cast in terms of a known, periodic forcing function, it can be solved analytically through double integration, resulting in a general solution and a particular solution with two integration constants defined by the boundary conditions of the system. These integration coefficients can be determined for analytical functions embedded in a data set (e.g., sine wave, step, ramp) resulting in unique values for a particular patient. These values are representative of the processing time of an individual and are a sensitive measurement of the individual's degree of brain function impairment. They may be compared to the same individual's previous values (i.e., during recovery), another individual's values, or those of a population norm to track changes in performance.

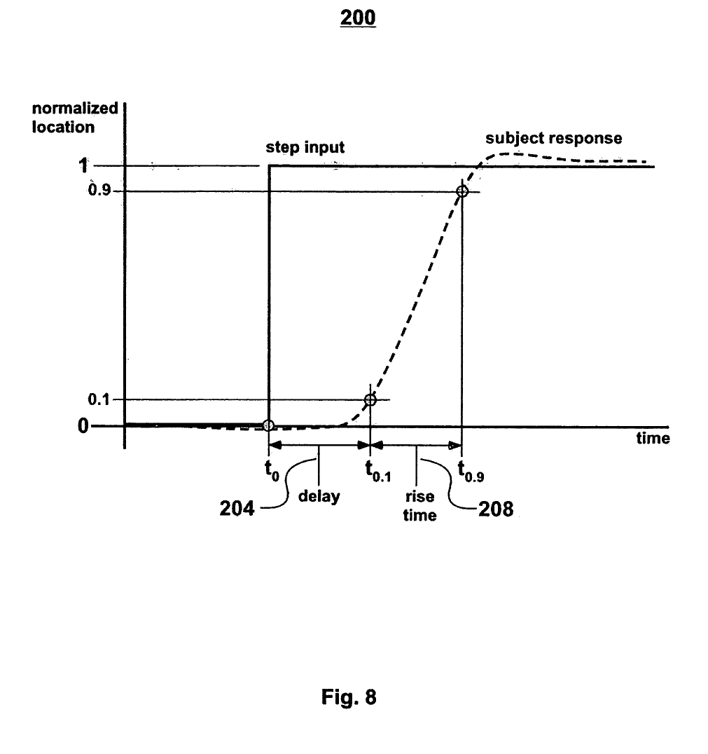

Referring now to FIG. 8, there is shown a graphical representation 200 of a target tracking response illustrating the delay time 204 and the rise time 208 of a test subject in the subject's response as the subject attempts to track the target 162 on the computer screen with the cursor 166. In this example, the response shown is for a step movement of the target 162, for example the step 168, the step 170 or the step 172 of the Target Tracking test 160.

Duhamel's Integral represents the solution to a second order linear system subject to an arbitrary, non-periodic forcing function:

$$u = \frac{{ⅇ}^{- nt}}{{ω}_{d}} {\int }_{0}^{t} {ⅇ}^{- {nt}^{′}} q sin {ω}_{d} ( t - {t}^{′} ) ⅆ {t}^{′}$$

This integral expression can be solved and numerically approximated to fit unique integration coefficients that relate the response of the patient to the entire set of prescribed motion of the target 162. In this way, the profile of the response of the patient is modeled mathematically and can be compared to statistical norms or the patient's own performance as recovery proceeds.

Individual features of the target tracking exercise may also be extracted in order to measure the subject's temporal reaction to spatial changes in position of the moving target 162. Again, the subject's response to a step input yields temporal measurements characteristic of second order linear systems. For example, the patient's response to a step input 168, 170, 172 can be extracted and analyzed for two temporal parameters indicative of functional ability: delay time 204 and rise time 208.

The nature of the delay time parameter 204, or the time it takes for the subject to recognize the instantaneous change in the position of the target 162 and initiate a movement to the new position, would intuitively place its duration between the simple reaction and choice reaction times previously described. The rise time parameter 208, or the time it takes to move from the pre-step position to the new target 162 position is a function of the visual-motor feedback control system responsible for locating the target 162 and moving to it. This parameter can complement the simple and choice reaction time measurements in determining brain function impairment in system 10.

A fifth proposed temporal parameter for computation of the PTI is Time In Target. The position of the cursor 162 may be compared with the position and extent in the target 162 to determine the percentage of the total target tracking test duration that was actually spent inside the target 162.

When computing the PTI each of the five temporal parameters can be normalized by the appropriate population normal to yield five ratios. Each of the five ratios can be compared to a value of one, meaning normal with respect to the population. A combination of these ratios can yield a single value termed the Processing Time Index (PTI) that can be compared to the normal value of one. In one embodiment of the invention, a simple arithmetic mean of the five ratios can be implemented to generate a PTI. As more data are collected, alternative algorithms for computing PTI can be evaluated, potentially implementing optimization methods to weight (higher or lower) each ratio based, for example, upon the magnitude of its variation with respect to the population normal.

The proposed PTI may therefore be expressed in the following form:

$$CSI = \frac{{\sum }_{i = 1}^{n} \frac{{x}_{i}}{{X}_{i}}}{n}$$

where xᵢ is the subject test score for the i^{th} test parameter, Xi is the population normal for the i^{th} test parameter and n=7 test parameters in the proposed battery: SRT (three modalities), CRT (three modalities), delay time 204, rise time 208 and time in target. The computed PTI can be compared to the population normal value, which, by default can yield a PTI of 1.0 for a “normal” patient or a value greater than one, designating potential impairment.

The range of PTI that can be designated as normal can be computed by evaluating the mean percentage standard deviation for each of the n tests: where σ_{Xi} is the standard

$$Range = \frac{{\sum }_{i = 1}^{n} \{ \frac{{σ}_{{X}_{i}}}{{X}_{i}} \}}{n}$$

deviation for the i^{th} test parameter and Xᵢ and n are defined as above. The term in brackets represents the ratio of the standard deviation to the mean for a given test parameter, or the percentage of the mean that the standard deviation represents for that test parameter.

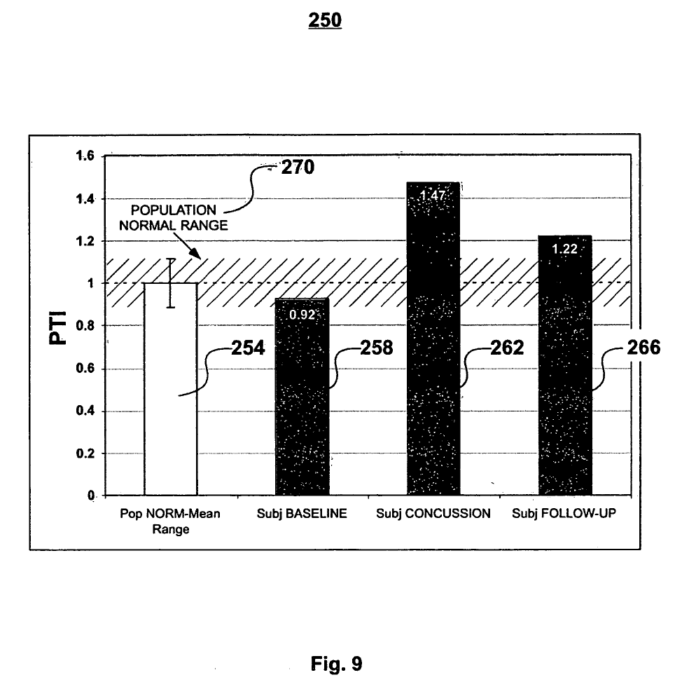

Referring now to FIG. 9, there is shown a bar graph 250. The bar graph 250 graphically illustrates a PTI 262 for an injured National Football League player compared to the National Football League population normal mean 254 and the population normal range 270. The PTI and population normal range 270 in this example were computed from previous National Football League data for n=3 available test modalities: visual SRT, visual CRT and rise time 208 from Target Tracking.

Implementing this algorithm for PTI in the context of the previous National Football League experience yields PTI values and the population normal range for the previously discussed National Football League concussion case study. The subject player's baseline PTI 258 is better than the population mean 254 and lies within the population normal range 270. Following the injury, the player's PTI 262 is elevated as expected and falls outside the computed normal population range 270. A follow-up examination PTI 266 demonstrates a trend back toward the pre-injury value of PTI 254, but still lies outside, albeit slightly, the population normal range 270. The baseline PTI 258 was calculated with the available National Football League test data, an n=3: SRT, CRT and rise time.

The bar graph 250 demonstrates the results of this analysis generated with the brain function impairment detection system 10 for a concussed player from the National Football League. Because all of the test parameters increase with impairment, a value greater than unity potentially indicates some impairment. In this example, the injured player can be considered to be approaching functional recovery with respect to the population norms as his PTI approaches the range 270 of the population's baseline, as it does in his follow-up exam.

Because the brain function impairment detection system 10 can be self-contained and operated by the end-user, i.e., medical professionals, it is useful to provide features that offer the end-user immediate feedback regarding the status of the subject or patient. An automated algorithm based upon the previously discussed formulation of the PTI can be applied to the administered test battery data to yield PTI immediately upon completion of the tests. This quantitative rating system can use a threshold score or continuous scale of impairment to automate the impairment rating and provide on-site personnel, such as trainers, medical techs, nurses and physicians, with immediate feedback. More detailed analysis and processing of the raw data may be performed for use of the system 10 as a research tool.

In addition to being a diagnostic tool, the brain function impairment detection system 10 functions as a research tool to elucidate quantitatively the functional effects of cerebral concussion (and other neurologic conditions) on subject impairment and recovery. A closer, more detailed, analysis of the full range of collected data permits the careful examination of the effects of cerebral concussion on specific neural function as well as correlation with experimental injury tolerance criteria for DAI previously determined. For example, Target Tracking results for a specific individual may be modeled as a second order linear system that, when compared to the input, may be fit with unique mathematical constants characterizing the individual test subject's performance. The change in those constants may be examined for injured patients not only to assess their level of impairment, but also to determine the specific effect of injury and recovery on the behavior of the analytical model. When this analytical tool is paired with knowledge of injury mechanism and injury tolerance, a broader portrait of the effect of mild traumatic brain injury on the structural and functional integrity and recovery of the central nervous system may be developed. To this end, the remote transfer of data from the end-user to a centralized research facility may be easily implemented via an Internet connection, enabling further study of accumulated data on impaired and normal subjects, and refinement of the PTI algorithm and normal and impaired statistical ranges for the population.

Thus, the impairment following a mild traumatic brain injury (i.e., concussion), the impairment due to aging, or the impairment due to intoxication can be measured through the examination of a patient's processing time changes using the brain function impairment detection system 10. The system 10 for characterizing impairment consists of active apparatus components that quantify processing time changes, that is, the patient must be conscious and able to interact with the system. The level of impairment of the patient is determined through direct comparison of his or her processing time changes to the normal processing time performance of the healthy population and a new measure of the impairment, known as the processing time index, is calculated.

The components of the brain function impairment detection system 10 are intended for clinical and scientific use both in a fixed, clinical setting and in mobile field use. This apparatus would provide the clinical professional (EMT, athletic trainer, emergency room physician, neuropsychologist and rehabilitation professional, etc.) with a flexible set of powerful tools to help measure, diagnose and treat mild traumatic brain injury and other neurological impairment more efficiently and cost-effectively.

Passive System (Ocular Assessment Tool Apparatus)

Another embodiment of the processing time index system is a hand-portable apparatus that measures the pupillary response of a patient and correlates specific temporal characteristics of that response to the processing time data and results generated with the computer based system 10. This embodiment is known as the Ocular Assessment Tool (OAT). Unlike the computer-based system 10, the hand-portable apparatus would be a passive system (i.e., not requiring direct user interaction); it is therefore useful on unconscious as well as conscious patients.

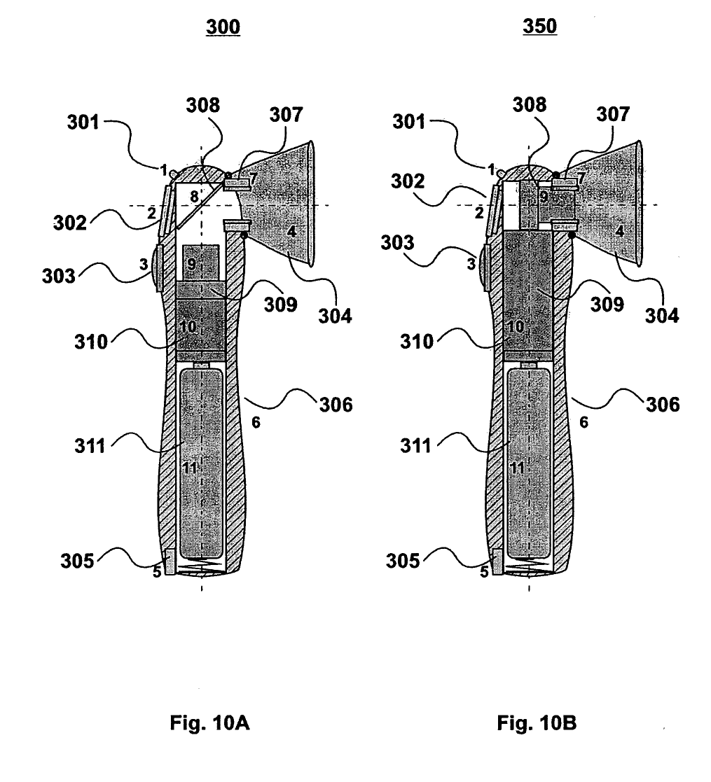

Referring now to FIGS. 10A,B, there are shown schematic representations of two alternate hand held embodiments 300, 350 of such a passive apparatus, the Ocular Assessment Tool (OAT). Many automated benchtop and hand held portable apparatuses exist that measure various aspects of pupillary response, or the contraction of the pupil upon increase in light intensity sensed on the retina, and can be used with the present invention. However, OATs such as the hand held OATs 300, 350 are also suitable for use in the present invention. Typical OATs include an indicator LED/battery low LED 301 and an LDC display panel 302. A button 303 for input/go/power is provided as well as a disposable eye cup 304. Features such as a mirror 308 and a camera 309 can also be provided. A processing module 310 for performing the algorithmic operations of, and correlating the OAT data with, the computer-based system 10 the brain function impairment system 10 and a battery 311 for energizing the system 10 are enclosed within a case 306.

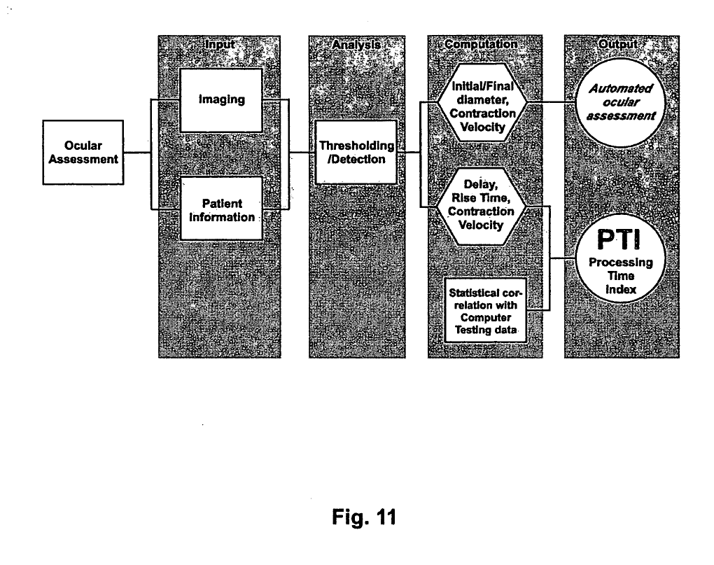

The OATs 300, 350 illuminate the eye resulting in the pupil of the eye contracting in response to the applied stimulus (light illumination). The system incorporates light stimulation and light imaging, but may also use light stimulation in concert with infrared imaging to decouple the effect of the stimulus on the imaging. The OATs 300, 350 can measure the initial diameter and the final diameter of the pupil, and can track the change in the diameter as a function of time. These parameters can also be operated upon to determine processing time, and thereby brain function impairment. These parameters can also be correlated with delay time 204 and rise time 208 in the computer system 10 to extend the methodology of the computer system 10 into a hand held, portable apparatus capable of determining PTI in an unconscious patient (FIG. 11). In addition, the OATs 300, 350 will deliver an automated version of the traditional pupillary reflex neurological examination and report the measured initial and final pupil diameters as well as the contraction velocity.

Although various automated bench top and hand portable apparatus exist to measure various aspects of the pupillary response, or contraction of the pupil upon increase in light intensity sensed on the retina, our apparatus extends the application of the traditional ocular assessment to incorporate standard measuring techniques into an algorithm for computing PTI. The system characterizes impairment following a concussion by measuring the change in a patient's pupillary response to light and comparing it to normal responses for the patient population. Additionally, the data collected and analyzed with the active, computer-based system may be correlated with pupillary response data (normal and impaired) to link the two methods of determining PTI into a cohesive, continuous and consistent method of assessing patient impairment by either method.

Input of patient-specific information, such as age, gender, etc., is necessary to compute PTI, due to the requirements of comparing the patient to the normal population. This information is stored along with the gathered test data for further processing. Patient privacy is preserved through an automatically generated anonymous identification number, as previously described for the computer-based system.

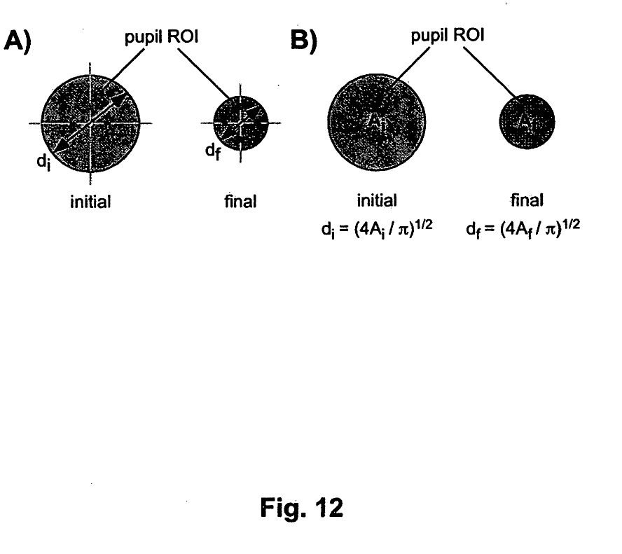

The system images the eye as the pupil contracts in response to the applied stimulus (light illumination). Image detection of the pupil is achieved by standard edge-finding, region-of-interest (ROI) image processing techniques. The diameter of the pupil is measured either a) directly, by locating the center of the pupil ROI and measuring the diameter across the ROI at several angles through this point; or b) indirectly, by measuring the area of the ROI and calculating a mean diameter (FIG. 12). The pupil diameter as a function of time is evaluated during the contraction period at a sampling rate on the order of 30 frames per second. The image and diameter data are stored for further analysis.

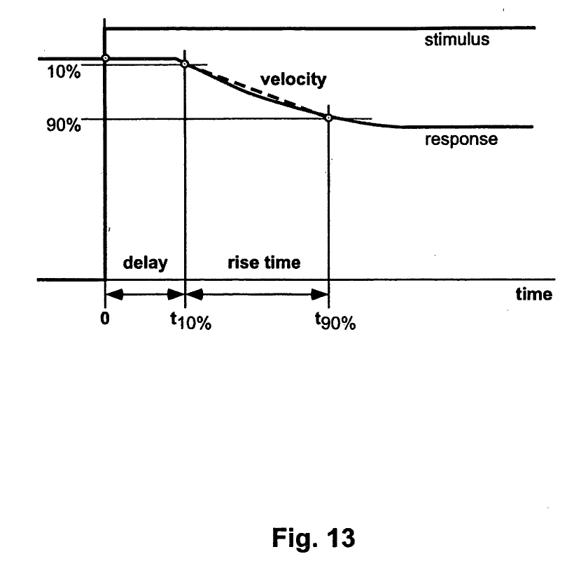

Once the pupil diameters are established as a function of time for the contraction event, the delay, rise time and contraction velocity parameters are calculated (FIG. 13). The delay and rise time parameters are determined in a manner similar to their counterparts in the computer-based target tracking exercise. All of the time-dependent data, as well as the calculated parameters, patient data and identification are stored for further analysis. PTI in the OAT is expressed as a function of time-dependent variables (processing time) in a manner similar to PTI determined in the computer-based system. Ultimately, PTI from the computer-based system can be statistically correlated to the PTI from the OAT to form a consistent, uniform PTI that is apparatus-independent.

While the invention has been described in detail and with reference to specific examples thereof, it will be apparent to one skilled in the art that various changes and modifications can be made therein without departing from the spirit and scope thereof.

1. A method for determining the severity of brain function impairment in a person due to an insult to said brain such as mild traumatic brain injury, aging or intoxication, comprising the steps of:
(a) determining a processing time value of said person in accordance with an elapsed time between a stimulus applied to said person and a response to said stimulus provided by said person, wherein said processing time value is representative of said impaired brain function;
(b) providing electrical signals in accordance with said processing time value, whereby said electrical signals are representative of said impaired brain function;
(c) performing mathematical operations upon said electrical signals to provide a processing time index; and
(d) determining said severity of said brain function impairment in accordance with said processing time index.

2. The method for determining the severity of brain function impairment in a person of claim 1, comprising the further step of determining concussion severity in accordance with said processing time index.

3. The method for determining the severity of brain function impairment in a person of claim 2, comprising the further step of determining said severity of said brain function impairment using pupillometry.

4. The method for determining the severity of brain function impairment in a person of claim 3, comprising the further step of determining said severity of said brain function impairment using a hand held pupillometer.

5. The method for determining the severity of brain function impairment in a person of claim 2, comprising the further steps of:
(a) applying a visual stimulus to said person; and
(b) determining said severity of said brain function impairment in accordance with said visual stimulus.

6. The method for determining the severity of brain function impairment in a person of claim 2, comprising the further steps of:
(a) applying an audio stimulus to said person; and
(b) determining said severity of said brain function impairment in accordance with said audio stimulus.

7. The method for determining the severity of brain function impairment in a person of claim 6, comprising the further steps of:
(a) applying a visual stimulus to said person; and
(b) determining said severity of said brain function impairment in accordance with both said audio stimulus and said visual stimulus.

8. The method for determining the severity of brain function impairment in a person of claim 7, comprising the further steps of:
(a) applying a somatosensory stimulus to said person; and
(b) determining said severity of said brain function impairment in accordance with said audio stimulus, said visual stimulus and said somatosensory stimulus.

9. The method for determining the severity of brain function impairment in a person of claim 2, comprising the further steps of:
(a) applying a somatosensory stimulus to said individual; and
(b) determining said severity of said brain function impairment in accordance with said somatosensory stimulus.

10. The method for determining the severity of brain function impairment in a person of claim 5, comprising the further steps of:
(a) determining a response to said visual stimulus from said person; and
(b) determining said processing time value in accordance with a time elapsed between the time of said applying of said visual stimulus to said person and the time of said determining of said response to said visual stimulus.

11. The method for determining the severity of brain function impairment in a person of claim 6, wherein the step of determining said processing time comprises the further steps of:
(a) determining a response to said audio stimulus from said person; and
(b) determining said processing time value in accordance with a time elapsed between the time of said applying of said audio stimulus to said person and the time of said determining of said response to said audio stimulus.

12. The method for determining the severity of brain function impairment in a person of claim 10, comprising the further steps of:
(a) applying an unfilled geometric shape to said person;
(b) applying a filled geometric shape to said person;
(c) determining a response to said filled geometric shape from said person; and
(d) determining said processing time value in accordance with a time elapsed between said applying of said filled geometric shape to said person and the time of said determining of said response to said filled geometric shape.

13. The method for determining the severity of brain function impairment in a person of claim 12, comprising the further steps of:
(a) applying a first plurality of unfilled geometric shapes to said person;
(b) applying a second plurality of filled geometric shapes to said person wherein said second plurality is smaller than said first plurality;
(c) determining a response to said applying of said second plurality of filled geometric shapes; and
(d) determining said processing time value in accordance with a time elapsed between said applying of said second plurality of filled geometric shapes to said person and said response to said applying of said second plurality of geometric shapes.

14. The method for determining the severity of brain function impairment in a person of claim 9, comprising the further steps of:
(a) determining a response to said somatosensory stimulus from said person; and
(b) determining said processing time value in accordance with a time elapsed between said applying of said somatosensory stimulus and said response to said somatosensory stimulus.

15. The method for determining the severity of brain function impairment in a person of claim 2, comprising the further step of determining said processing time value in accordance with a tracking of the motion of a moving target by said person.

16. The method for determining the severity of brain function impairment in a person of claim 15, comprising the further step of determining said processing time value in accordance with the ability of said person to track said motion of said moving target with a cursor controlled by a manual control device when said moving target is displayed on a video display.

17. The method for determining the severity of brain function impairment in a person of claim 15, wherein said motion of said moving target comprises a step function stimulus having a predicted response and an elicited response from said person.

18. The method for determining the severity of brain function impairment in a person of claim 17, wherein said elicited response comprises a damped oscillatory response.

19. The method for determining the severity of brain function impairment in a person of claim 18, comprising the further step of determining a delay time value in accordance with said damped oscillatory response.

20. The method for determining the severity of brain function impairment in a person of claim 19, comprising the further step of determining a rise time value in accordance with said damped oscillatory response.

21. The method for determining the severity of brain function impairment in a person of claim 20, comprising the further step of determining said processing time value in accordance with said delay time value and said rise time value.

22. The method for determining the severity of brain function impairment in a person of claim 16, comprising the further steps:
(a) disposing said cursor within said target
(b) applying a forcing function to said target to cause said target to move on said display;
(c) instructing said person to use said manual control device to move said cursor on said display and maintain said cursor within said moving target while said target is in motion; and
(d) determining said processing time value in accordance with the amount of time said person maintains said cursor within said moving target.

23. The method for determining the severity of brain function impairment in a person of claim 2, comprising the further step of comparing said processing time index with a value representative of a population norm.

24. The method for determining the severity of brain function impairment in a person of claim 23, comprising the further step of determining a status of said person in accordance with said comparing.

25. The method for determining the severity of brain function impairment in a person of claim 2, comprising the further step of comparing said processing time value with a previous processing time value of said individual.
